(19) United States
(12) Patent Application Publication
(10) Pub. No.: US 2025/0235690 A1
(43) Pub. Date: Jul. 24, 2025

(54) REHYDRATABLE ELECTRODE FOR NEUROSTIMULATION

(71) Applicants: Douglas Richard Jeffrey, Oakland, CA (US); Shriram Raghunathan, Castro Valley, CA (US)

(72) Inventors: Douglas Richard Jeffrey, Oakland, CA (US); Shriram Raghunathan, Castro Valley, CA (US)

(21) Appl. No.: 18/702,139

(22) PCT Filed: Oct. 20, 2022
(86) PCT No.: PCT/US2022/047239

§ 371 Date: Apr. 17, 2024

Related U.S. Application Data
(60) Provisional application No. 63/270,022, filed on Oct. 20, 2021.

(51) Int. Cl. A61N 1/04; B05B 17/06

(52) U.S. Cl. CPC A61N 1/0496; A61N 1/0456; A61N 1/048; B05B 17/06

(57) ABSTRACT

A neurostimulation system can include a skin electrode pad configured to be attached to an external electrostimulation electronics unit for treating a subject using transcutaneous neurostimulation therapy, and the electrode pad can include a rehydratable hydrophilic foam layer. The rehydratable hydrophilic foam layer can be sized and shaped to be received within a receptacle of an electrode rehydrator to recurrently receive moisture from the electrode rehydrator to be rehydrated.

### CLAIM OF PRIORITY

This application claims priority to U.S. Provisional Application Ser. No. 63/270,022, filed on Oct. 20, 2021, which is incorporated by reference herein in its entirety, and the benefit of priority of which is claimed herein.

### TECHNICAL FIELD

This document pertains generally, but not by way of limitation, to skin surface electrodes, and more particularly to systems and methods for recurrently rehydrating or rewetting neurostimulation skin surface electrodes such as to help increase the lifespan or effectiveness of the electrodes.

### BACKGROUND

Skin surface electrodes are important components in many medical systems including, e.g., functional stimulation electrical (FES), electrocardiogramalectromyography (EMG), electroencephalography (EEG), or electrooculography (EOG). Such electrodes can provide transcutaneous delivery of electrical neurostimulation or can act as sensing or recording electrodes for corresponding transducers to measure, e.g., biopotentials such as for diagnostic testing. The workings of skin surface electrodes are dependent on a multitude of mechanisms, such as electrode material, electrolyte applied to the electrode, body location, and skin properties.

### SUMMARY

In several approaches using skin surface electrodes, a hydrogel electrode pad can be used in a medical system for transcutaneous electrical contact with the body. The hydrogel can contain electrolyte to help the hydrogel electrode pad conduct electricity, and the hydrogel can adhere to skin for a procedure. A challenge with this approach, however, is that the hydrogel can become dehydrated which can cause the hydrogel to lose some of its conductive or adhesive properties. Here, the hydrogel electrode pads must be frequently replaced for therapy to maintain acceptable electrode impedances or to ensure proper contact with the skin surface. The present inventors have recognized, among other things, the need for an electrode pad or system which is more resilient to electrode pad degradation to provide consistent, effective treatment. The present inventors have also recognized that including electrode rehydration with a docking station such as also for device charging can help enable maintenance and care of electrodes and reduce a need to disassemble components of the system.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A depicts a perspective view of an example of a transcutaneous neurostimulation therapy system.

FIG. 1B depicts a perspective view of a transcutaneous neurostimulation therapy system.

FIG. 2A depicts an example of a wearable electrostimulation device in use with a subject.

FIG. 2B depicts an example of a wearable electrostimulation device in use with a subject.

FIG. 3A depicts a perspective view of an example of skin electrode pads being used with an electrostimulation electronics unit.

FIG. 3B depicts a perspective view of an example skin electrode pad.

FIG. 3C depicts a perspective view of an example skin electrode pad.

FIG. 4 is a schematic representation of an example of a transcutaneous neurostimulation therapy system.

FIG. 5 is a schematic representation of an example of a transcutaneous neurostimulation therapy system.

FIG. 6 is a schematic representation of an example of a transcutaneous neurostimulation therapy system.

FIG. 7 is a schematic representation of an example of a transcutaneous neurostimulation therapy system.

### DETAILED DESCRIPTION

This document describes, among other things, skin surface electrodes, and more particularly to systems and methods for recurrently rehydrating or rewetting skin surface neurostimulation electrodes such as to help increase one or both of the lifespan or effectiveness of the electrodes. Skin surface electrodes can be used to perform a neurostimulation treatment or diagnostic procedure such as a part of a medical system for delivering electrical neurostimulation or recording electrical response activity. For example, electrodes can be used with an electrostimulation electronics unit for delivering transcutaneous neurostimulation. Here, electrical impulses can be delivered from the electrodes such as to mimic or elicit a neural action potential. For example, for pain therapy, electrical impulses can be delivered below a motor-threshold such as in a manner such that muscles do not contract as a result of the electrical impulses. In other approaches, such as physical rehabilitation, physical therapy, or muscle recovery techniques, electrical impulses can be delivered above a motor-threshold, such as in for causing the muscles to contract.

The electrodes can include or use an electrode contact, such as can be surrounded by a hydrogel pad. The hydrogel can carry or contain electrolyte, such as to help the hydrogel electrode pad conduct electricity, to help hydrogel adhere to skin for the procedure, or both. Largely composed of water, hydrogel should remain moist or wet, such as to help ensure good electrical conductivity (reduced electrical resistance) at the skin-electrode interface and sufficient adhesion to the skin. Reduced moisture, such as either through drying out or “fouling” of the hydrogel by dirt, skin oils, dander, or other contaminants can result in reduced electrical conductivity (poor performance) and reduced adhesion (unsafe condition). In an extreme example, degraded hydrogel can lead to higher current density localized arcing and resulting discomfort such as may result from non-adhesion of the electrode to the skin. This can result in a need for frequent replacement of the hydrogel pads, which can be costly or difficult to achieve, particularly in a home treatment setting. The present systems and techniques can help reduce or prevent such discomfort related to electrostimulation and can help improve electrode longevity and performance.

The present disclosure relates to, among other things, systems and methods enabling delivering transcutaneous neurostimulation to a subject. The present disclosure also relates to a neurostimulation electrode pad that can be recurrently rehydrated, such as by a rehydrating apparatus. The system can help reduce the chance for user error or incorrect maintenance of electrodes. For example, the electrode pad can retain moisture and can be rehydrated with water vapor. The system can include or use an electrostimulation electronics unit that can detect a condition of the electrode pad, such as to help ensure proper electrode maintenance. The system can also include an electrode rehydrator, which can help rehydrate the electrode pad, such as based on an indicator, a water content of the electrode pad, or an impedance of the electrode pad. The electrode rehydrator can be a component of a docking station for storing the electrostimulation electronics unit absent use for electrostimulation. The electrostimulation electronics unit can be placed with the electrodes facing the docking station such as to receive both rehydration and battery recharging from the docking station, such as concurrently or at least during the same docking session.

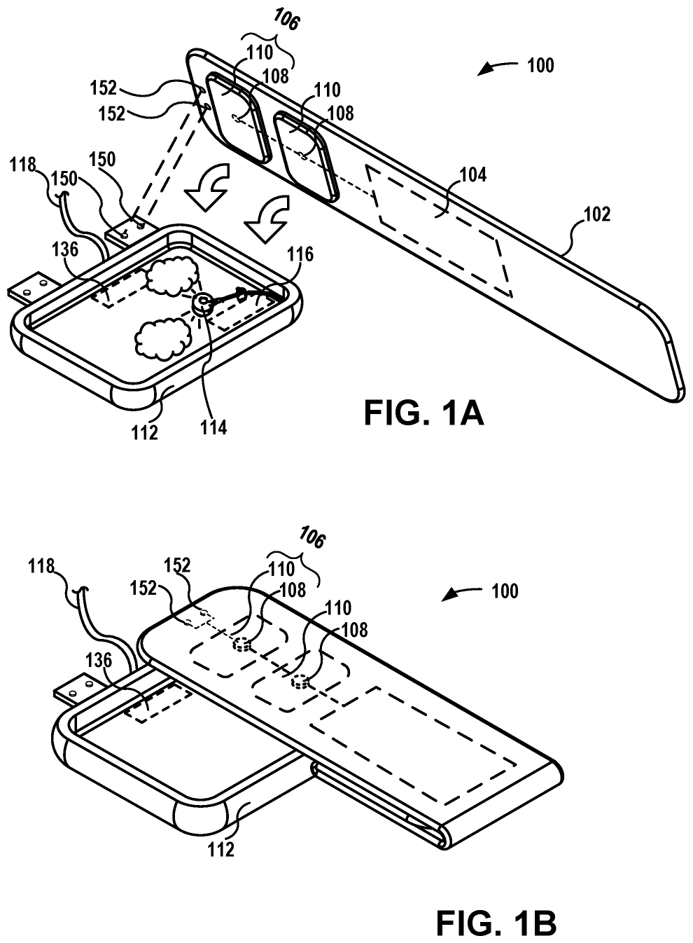

FIG. 1A and FIG. 1B show a perspective view of an example of a transcutaneous neurostimulation therapy system 100. In an example, the electrostimulation therapy system 100 can include a wearable electrostimulation device 102, an electrostimulation electronics unit 104, one or more electrodes 106, a device docking station 112, an electrode rehydrator 114, rehydrator control circuitry 116, and a power supply line 118. The electrostimulation therapy system 100 can function to deliver neurostimulation therapy to skin of a subject via the electrodes 106, such as over one or multiple therapy sessions and the present rehydration capability can help mitigate potential degradation of the electrode pad despite its repeated use.

The wearable electrostimulation device 102 can be worn by the subject and can include or use the electrostimulation electronics unit 104 coupled to the electrode 106, such as for transcutaneously delivering a neurostimulation signal. Herein, the term neurostimulation can include, for example transcutaneous electrical nerve stimulation (TENS), electrical muscle stimulation (EMS), electrical stimulation (e-stim), or other electrical neurostimulation. The wearable electrostimulation device 102 can be sized and shaped to be able to be attached or held to a body location of the subject, e.g., a leg, arm, foot, waist, neck, head, or chest of the subject. For example, FIGS. 1A, 1B show an example of a wearable electrostimulation device 102 that can include or use a strap to help hold the electrodes 106 to the skin of the subject. While electrodes are generally described herein with a focus on providing electrostimulation to a subject, the electrodes can alternatively or additionally be used to help detect or measure one or more biosignals or biopotentials from the subject. A particular electrode 106 can include or use an electrode terminal 108 and an electrode pad 110. For example, the electrode terminal 108 can receive a capacitively-coupled (e.g., coupled using series DC-blocking capacitors, e.g., in a charge-balanced arrangement) electrostimulation signal from the electrostimulation electronics unit 104, and can deliver a resulting neurostimulation signal to the skin of the subject, such as via the electrode pad 110. In an example, multiple electrodes, such as two electrodes 106, can be used. For a bipolar electrode example having two electrodes 106, this can include or use a first electrode that can serve as an anode and a second electrode, such as which can serve as a cathode. Also, a plurality of electrodes 106 can be arranged to form a multi-electrode group, matrix, or array such as for one or both of sensing or for delivering the electrostimulation signal to the skin of the subject. In an example, each electrode terminal 108 can include an electrode contact fixed to the wearable electrostimulation device 102 and each corresponding electrode pad 110 can be removably coupled to the device 102. In an example, the electrode pad can be fixed to the wearable electrostimulation device 102. The electrode terminal 108 can carry an alternating current (AC) electrostimulation signal for delivery to the skin by the electrode 106. For example, the electrostimulation signal can be provided by the electrode terminal 108 at a frequency between about 4 kHz to about 15 kHz, such as for treating Restless Leg Syndrome (RLS) or Periodic Limb Movement Disorder (PLMD), such as described in Charlesworth U.S. Pat. No. 11,103,691, which is hereby incorporated by reference herein. The wearable electrostimulation device 102 can include or use capacitive coupling such as with DC block capacitors in series with the electrode terminal 108, such as to block a DC signal and pass an AC signal. The AC electrostimulation signal can be delivered from the electrode terminal 108 to the skin through the electrode pad 110 disposed therebetween.

The electrode pad 110 can include or use an electrically conductive layer such as to help create a stable electrode-skin interface that can retain its ability to deliver appropriate and targeted signals in a manner that does not significantly change over time. Because the current density of the neurostimulation signal at the electrode terminal may be larger than desired, the electrode pad may include an embedded or other arrangement of electrical conductors that can help distribute the electrostimulation signal current over a larger effective surface area for delivery to the subject at the skin-electrode interface. This can help disperse stimulation charge across a larger surface area of the skin and thus can help to reduce or prevent subject discomfort. The substantially higher charge transfer area introduced by the electrode pad 110 can help lower current density and allow neurostimulations to be delivered more consistently and at higher voltages and lower power consumption. The conductive layer of the electrode pad 110 can include or use an absorbent material. The absorbent material can be capable of receiving vapor from a mister, humidifier, or other apparatus capable of producing vaporized liquid. The absorbent material can be capable of receiving liquid from an electrode rehydrator 114, such as can include one or more of a reservoir, injector, or sprayer.

In an example, the wearable electrostimulation device 102 can be sized and shaped such as to be interfaced with or be received within a receptacle of a device docking station 112. As shown in FIG. 1A and FIG. 1B, the wearable electrostimulation device 102 can be placed with the electrodes 106 facing the docking station 112 such as to place the electrodes 106 in proximity with the electrode rehydrator 114 of the docking station 112. As shown in FIG. 1B, at least a portion of the wearable electrostimulation device 102 can be folded, rolled, or otherwise compacted such as to help minimize the space required for device docking. In an example, the electrode pad 110 can be removed from the wearable electrostimulation device 102, and the electrode pad (or multiple electrode pads) alone can interface with the docking station 112 such as for storing or rehydration. As shown in FIG. 1A and FIG. 1B, the docking station 112 can be sized and shaped such as to be capable of receiving more than one (e.g., a pair of) wearable electrostimulation devices 102 such as for providing a receptacle or reservoir or other docking-station location configured for electrode rehydration. For example, the wearable electrostimulation device 102 can be placed with the electrodes 106 facing the docking station 112 such as to receive both rehydration and battery recharging from the docking station 112, such as concurrently or at least during the same docking session. Battery charging circuitry 136 can be included near the electrode rehydrator 114 such within the docking station 112. The battery charging circuitry 136 can generate a charging signal, and the charging signal can be carried through charge-supplying electrical contacts 150. Charge-receiving electrical contacts 152 of the wearable electrostimulation device 102 can be connected to the charge-supplying electrical contacts 150 at the docking station 112 for device charging concurrent with electrode rehydrating from the rehydrator 114. In an example, the charge-receiving electrical contacts can be the electrode terminals 108. In another example, the charge-receiving electrical contacts can be the skin contact surface of the electrode pad 110, and the docking station 112 and wearable electrostimulation device 102 can include or use circuitry for charging the device through, e.g., a hydrophilic polymer material of the electrodes 106, such as described in Colborne U.S. Provisional Patent Application Ser. No. 63/270,011 entitled REVERSE ELECTRODE CHARGING FOR NEURAL ELECTROSTIMULATION, (Attorney Docket No. 4991.009PRV), filed on even date herewith, which is incorporated by reference herein in its entirety. In an example where the battery charging circuitry 136 can charge the battery of the electrostimulation electronics unit 104 through the electrode pads 110 while the electrode pads are concurrently rehydrated by the electrode rehydrator 114, the docking station 112 can provide an electrically isolated barrier between electrode pads. For example, the docking station can provide an electrically isolated barrier between charge-supplying electrical contacts 150, and each electrode pad 110 can be sized and shaped such as to be placed in an electrically isolated receptacle created by the barrier.

The docking station 112 can include or use an electrode rehydrator 114, rehydrator circuitry 116, a power supply line 118. The electrode rehydrator 114 can be at least partially disposed in an enclosure or body of the docking station 112, and the enclosure or body can include, e.g., a basin, reservoir, or a surface with perforations or a grid of holes. The electrode rehydrator 114 can include or use one or more vaporizers configured to vaporize a liquid. One of the electrode rehydrator 114 or the docking station 112 can include or use a reservoir for storing liquid to be vaporized. In an example, the liquid to be vaporized is water such as hard water or distilled water. In another example, the liquid to be vaporized includes electrolyte. The liquid to be vaporized can also include an alcohol or saline solution. In an example, the vaporizer can be an include or use a heating element for boiling liquid to create steam as the vapor. In another example, the vaporizer can include an ultrasonic humidifier. The ultrasonic humidifier can include or use a ceramic diaphragm that vibrates at an ultrasonic frequency using, e.g., one or more piezoelectric transducers. Vibration of the liquid at the ultrasonic frequency can create small water droplets or vapor. In yet another example, the liquid can be sprayed through a nozzle e.g., at a high pressure or with compressed air. Here, the nozzle can be formed or machined such that liquid traveling therethrough vaporizes and the heat of vaporization is absorbed by the surrounding air. The average droplet diameter of the liquid created by the vaporizer can be within a range of about 0.25 cubic microns to about 65,500 cubic microns. In another example, the average droplet diameter of the liquid created by the vaporizer can be within a range of about 0.25 cubic microns to about 0.75 cubic microns. In yet another example, the average droplet diameter of the liquid created by the vaporizer can be within a range of about 0.50 cubic microns to about 0.55 cubic microns. In some examples, the electrode rehydrator 114 can be connected to rehydrator circuitry 116, and the rehydrator circuitry 116 can function to selectively operate e.g., the vaporizer of the rehydrator 114 during docking of the wearable electrostimulation device 102. In another example, the electrode rehydrator 114 can include an apparatus, e.g., a reservoir, liquid injector, or liquid sprayer for distributing liquid directly to the surface of the electrode pad 110. In yet another example, liquid can fill the docking station basin, and the liquid can be absorbed by the electrode pad 110 placed therein.

As described in greater detail below, the rehydrator circuitry 116 can communicate with or detect presence of the wearable electrostimulation device 102 or the electrode pad 110 such as to determine a device docking status. The rehydrator circuitry 116 can operate the electrode rehydrator 114 when the electrode pad 110 is in proximity to the electrode rehydrator 114. In an example, the rehydrator circuitry 116 can operate the electrode rehydrator 114 when the wearable electrostimulation device 102 is interfaced with the docking station 112. In some examples, either of the electrode rehydrator 114 or the rehydrator circuitry 116 can be connected to an energy source. The energy source can be a battery onboard the docking station 112, or the energy source can be an external source of power (e.g., AC power receptacle) connected by the power supply line 118.

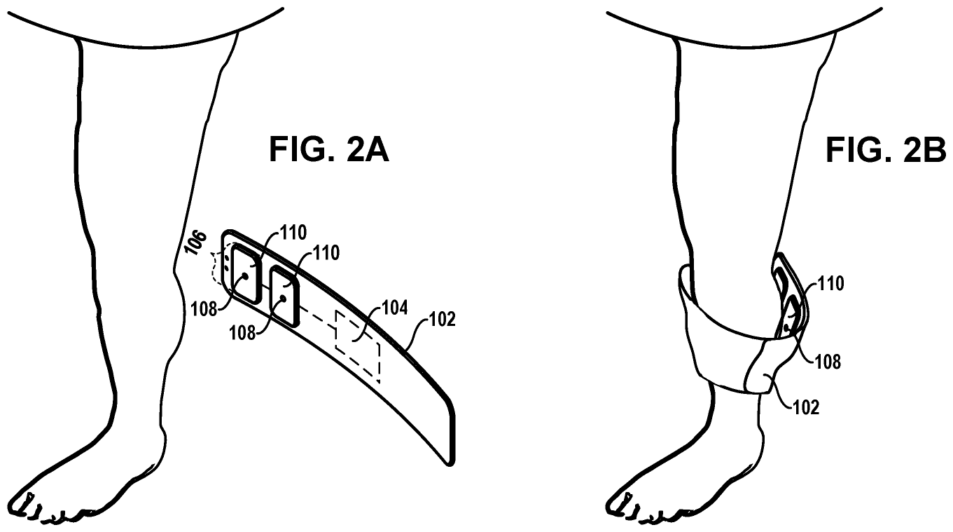

FIG. 2A and FIG. 2B show an example wearable electrostimulation device in use on a subject. In an example, an electrostimulation therapy system 100 can include a wearable electrostimulation device 102, an electrostimulation electronics unit 104, and one or more electrodes 106. The electrostimulation therapy device 102 can function to deliver neurostimulation therapy to skin of a subject via the electrodes 106, and the device can be capable of delivering such therapy over multiple therapy sessions. As depicted in FIG. 2A & FIG. 2B, the wearable electrostimulation device 102 can include or use a strap, sleeve, band, or clamp to help hold the electrodes 106 to the skin of the subject. In another example, the electrostimulation device can include or use an adhesive or can connect to other items wearable by the subject, e.g., hats, clothing, etc. Alternatively, the wearable electrostimulation device 102 can be sufficiently wearable on the skin surface of the patient by adhesion forces of the electrodes 106 alone without the need for additional features to help hold the device 102 to the subject. The wearable electrostimulation device 102 can be attached or held to a body location of the subject, e.g., a leg, arm, foot, waist, neck, head, or chest of the subject at or near a nerve location of the patient skin for transcutaneous neurostimulation thereof. The wearable electrostimulation device 102 can include or use the electrostimulation electronics unit 104 for producing an electrostimulation signal which can be distributed to the skin surface of the subject by the electrodes 106. The electrostimulation signal delivered to the skin surface of the subject by the electrodes 106 can be a charge-balanced or alternating current (AC) signal. In an example, the electrostimulation electronics unit 104 can include capacitive-coupling circuitry such as capacitors in series for distributing alternating current (AC) signal to the skin surface of the subject without distributing significant amounts of direct current (DC) signal to the skin surface of the subject. In an example, the wearable electrostimulation device 102 can be e.g., fastened to a body location of the subject for a transdermal neurostimulation therapy session. The electrostimulation therapy session can be performed for a predetermined period of time. The electrostimulation therapy session can be a recurrent therapy session, such as can be included as a member within a larger schedule of electrostimulation therapy sessions. In an example, the electrostimulation therapy session can be repeatable. In examples where multiple electrostimulation therapy can be performed, the wearable electrostimulation device 102 can be placed or stored at a docking station (as depicted in FIG. 1B) between some of the therapy sessions such as to help rehydrate the electrodes 106 or to help charge a battery of the electrostimulation electronics unit 104.

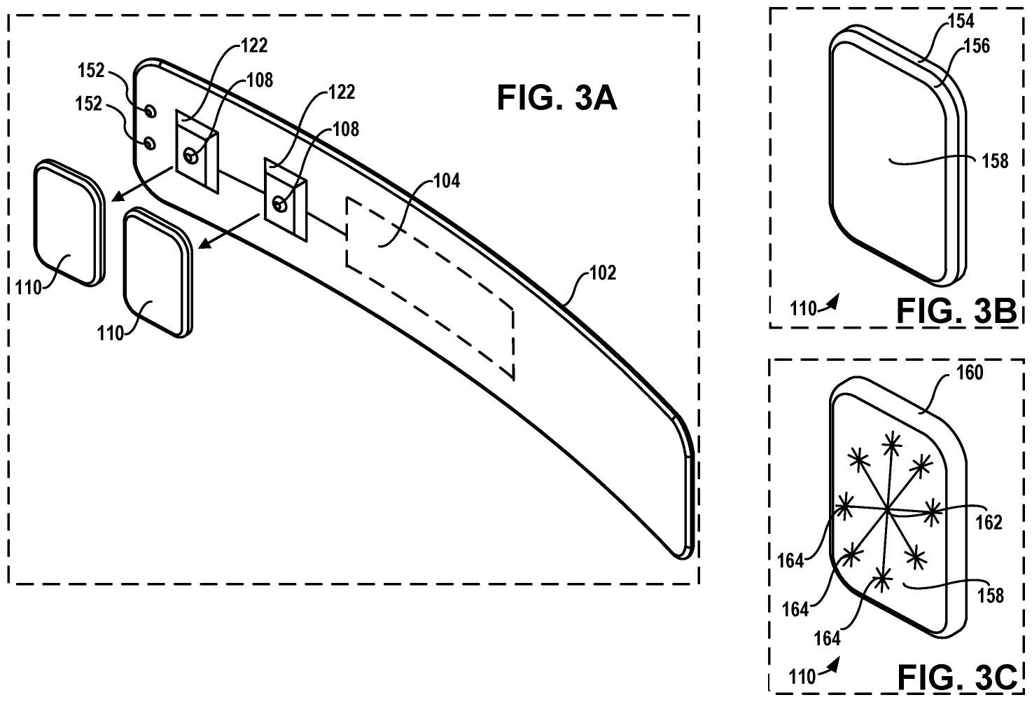

FIG. 3A shows a perspective view of an example of skin electrode pads being used with an electrostimulation electronics unit of a wearable electrostimulation device. In an example, an electrostimulation therapy system 100 can include a wearable electrostimulation device 102, an electrostimulation electronics unit 104, and one or more electrodes 106. The wearable electrostimulation therapy device 102 can function to deliver neurostimulation therapy to skin of a subject via the electrode pads 110 which help make up the electrodes 106 (as depicted in FIG. 1A). In an example, the electrode pads 110 can be removably coupled to the wearable electrostimulation device 102. The electrode pads 110 can each be attached to a pairing surface 122 of the wearable electrostimulation device 102. The pairing surface 122 can include the electrode terminal 108 e.g., disposed therein, and the electrode terminal 108 can be electrically connected to the electrostimulation electronics unit 104. In an example, as depicted in FIG. 3, two or more electrode pads 110 are each paired to corresponding pairing surfaces 122 including one electrode terminals 108. In another example, one electrode pad 110 can be paired to an electrode pairing surface 122 containing more than one electrode terminal 108, or one electrode pad can span multiple electrode pairing surfaces 122 containing one or more electrode terminals 108. The electrode pad 110 can be removed for, e.g., hygienic maintenance, electrode maintenance such as rehydrating, or disposal. As discussed further herein, the electrode pads can be removed based upon, e.g., detection of an electrode pad condition by the electrostimulation electronics unit 104.

FIG. 3B and FIG. 3C show a perspective view of an example electrode pads 110. The electrode pad 110 can include or use a conductive layer 154 formed at least in part by an absorbent material. The absorbent material can also form a separate absorbent layer 156, as depicted in FIG. 3B. The absorbent material can have a water contact angle ranging between about 0° and about 90°. In another example, the absorbent material can have a water contact angle ranging between about 40° and about 80°. In yet another example, the absorbent material can be hydrophilic, such as having a water contact angle ranging between about 45° and about 55°.

The absorbent material can help retain an electrically conductive liquid solution, such as a solution containing electrolyte, or a saline solution and the absorbent material can help retain moisture within the electrode pad 110. Electrolyte can be carried, disposed, or contained within the absorbent material. The absorbent material can include, for example, a hydrophilic polymer such as polyvinyl alcohol (PVA). One or more other hydrophilic polymers, such as carboxypolyalkylene, polyethylene oxide, polyoxyethylene-polyoxypropylene copolymer, hydroxypropyl, hydrophilic polyurethane, cellulosic, hydrophilic polyamide, polyvinyl pyrrolidone, hydrophilic acrylic, or hydrophilic silicone elastomer can be used to help form the absorbent material. In an example, the hydrophilic polymer can include or be made up of a polymer chain with polar end groups such as alcohol end groups. Also, copolymerizations including one or more combinations of hydrophilic polymers such as those listed above can be used to help form the absorbent material. The absorbent material can also be formed of cotton, cellulose, textiles, or types of conductive or dielectric gels. The absorbent material can be porous or formed into a foam or sponge material or can include multiple layers, such as can have different characteristics. For example, the absorbent material can be hypo-allergenic or bactericidal. The absorbent material can be at or near a skin contact surface 158 of the electrode pad 110, and the skin contact surface 158 of the electrode pad 110 can interface with or adhere to the skin of the subject. The absorbent material, where a part of the electrically conductive layer 154, can help deliver the electrostimulation signal from the electrode terminal and to the skin contact surface. The absorbent material can be capable of being repeatedly rewetted or rehydrated by the electrode rehydrator 114 such as to recurrently place or return the electrode pad 110 to one or more desired conditions (e.g., acceptable impedance, acceptable skin adherence, or acceptable conformal compliance of the electrode pad 110 to the skin). The absorbent material can quickly absorb moisture. In an example, the absorbent material can become at least 80% saturated after 20 seconds of contact with liquid water. The absorbent material can be recurrently or periodically rewetted or rehydrated, such as by a user, such as to allow repeated using of the same electrode pad 110 for multiple sessions of neurostimulation without significant degradation of the electrode pad 110 or significant reduction of the capabilities of the electrode pad 110 to deliver the electrostimulation signal for effective neurostimulation therapy. The electrode pad 110 can be relatively thin, such as having a thickness of a range between about 1/16 inch and about 3/16 inch while being able to deliver the electrostimulation signal safely, comfortably, and adequately to the skin. For a porous absorbent material, the thickness of the electrode pad 110 can be expressed as a proportion to a mean pore diameter of the absorbent material of the pad 110. For instance, the thickness of the electrode pad 110 can be within a range of about 5% and about 15% of the mean pore diameter of the absorbent material. The electrically conductive layer 154 can include one or multiple absorbent materials e.g., laminated or otherwise adjacently layered or otherwise connected to one another. Also, as depicted in FIG. 3B, the electrode pad 110 can include or use a separate absorbent layer 156 formed of the absorbent material. For example, the electrically conductive layer 154 can include a hydrophilic polymer such as a hydrogel as an interior layer laminated or otherwise formed adjacent to the absorbent layer 156, which can be another hydrophilic polymer such as a PVA foam and can include the outer subject contact surface 158. Here, moisture can migrate between the electrically conductive layer 154 and the absorbent layer 156, and the absorbent layer 156 can rehydrate the conductive layer 154.

In an example depicted in 3C, the electrode pad can include or use a substrate layer 160, such as can be embedded, impregnated, or lined with one or more electrically conductive traces 162. The substrate layer 160 can be formed of the absorbent material or can be laminated or connected an absorbent layer 156 (as depicted in FIG. 3B) formed of the absorbent material. Alternatively, the substrate layer 160 can be formed of a non-conductive or dielectric material. In an example, the substrate layer 160 is formed of polyethylene or another thermoplastic polymer. The substrate layer 160 can be flexible such as to conform to anatomy of the subject. The one or more electrical traces 162 can provide an electrical connection between either of the electrode terminal 108 and the skin contact surface 158. Furthermore, a mechanical connector can be included or used such as to help make an electrical connection between the electrode pad 110 and the electrode terminal 108. Some examples of the mechanical connector can include a snap connector, a pogo pin, or a plug/socket. The electrical trace 162 can be printed or applied to the substrate layer 160. For example, the trace 162 can be laser printed using conductive ink, flexographically printed, silk screened, etched, or soldered to the substrate layer 160. For example, a single trace 162 can be included in the electrode pad 110 such as to help deliver electrical current from the electrode terminal 108 toward the skin of the subject. In an example, a plurality of electrical traces 162 can be included in the electrode pad 110 such as to provide a dispersed region or multiple regions 164 for electrostimulation. Optionally, the multiple regions 164 can be separately or independently selectively controlled such as to enable selection of one or more desired regions 164 for electrostimulation.

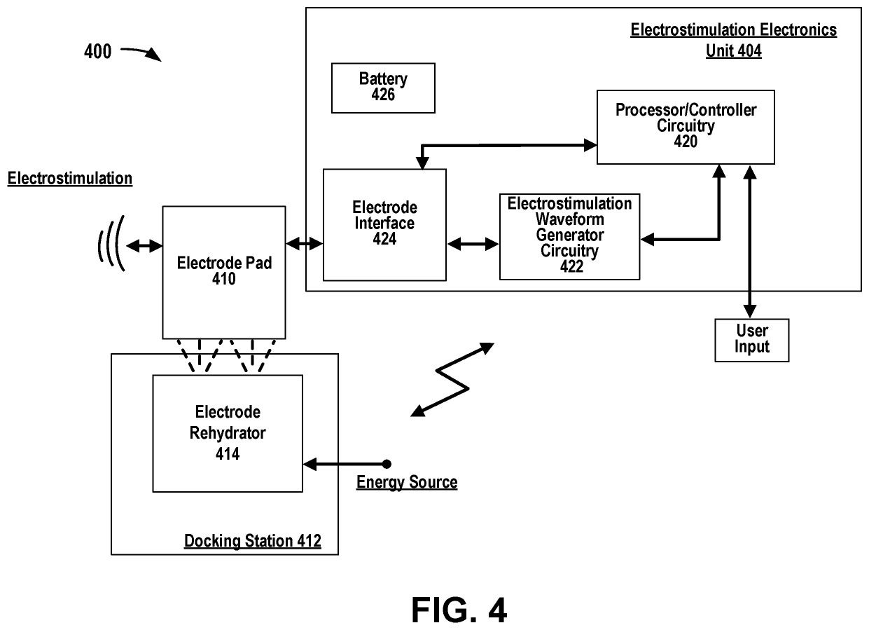

FIG. 4 is a schematic representation of an example of a transcutaneous neurostimulation therapy system. An example electrostimulation therapy system 400 can include an electrostimulation electronics unit 404, one or more electrode pads 410, a docking station 412 and an electrode rehydrator 414. The electrostimulation therapy system 400 can be similar in many respects to electrostimulation therapy system 100. The components, structures, configuration, functions, etc. of system 400 can therefore be the same as or substantially similar to that described in detail above with reference to system 100. The electrostimulation therapy system 400 can include the electrostimulation electronics unit 404 capable of communication with either of the docking station 412 or the electrode rehydrator 414. The electrostimulation electronics unit 404 can include or use a processor or controller circuitry 420, an electrostimulation waveform generator circuitry 422, an electrode interface 424, and a battery 426. The processor circuitry can receive a user input such as communications from a user interface (UI). The UI can include or use switches, buttons, knobs, touch panels, status LEDs, or display screens such as to enable user interaction for performing electrostimulation therapy. The display, status LEDs, or other similar components can be capable of displaying or indicating user data, test outcomes, or instructions. In one example the display can be an LCD screen embedded in either of the wearable electrostimulation device of the electrostimulation electronics unit 404. Alternatively or additionally, the user can interact with the electrostimulation electronics unit 404 by means of the software application on a remote device such as a computer or a mobile phone.

The processor circuitry 420 can be communicatively coupled with the electrostimulation waveform generator circuitry 422. The electrostimulation waveform generator circuitry 422 can be selectively operated by the processor circuitry 420 such as to provide an electrostimulation signal to the electrode interface 424. The electrostimulation signal can be generated by the generator circuitry 422 at a frequency within a range of about 4 kHz to about 15 kHz and the waveform can be sinusoidal. The electrode interface 424 can supply the electrostimulation signal to the electrode pads via, e.g., electrostimulation output terminals. In an example, the electrode interface 424 can be communicatively coupled to the processor circuitry 420 such as to provide feedback or instructions.

The electrode rehydrator 414 can be at least partially disposed within a docking station 412. The electrode rehydrator 414 can be connected to an energy source, and the rehydrator 414 can function to provide moisture to one or more electrode pads 410. The electrode rehydrator 414 can be capable of communication with the electrostimulation electronics unit 404 such as to help moderate electrode rehydration from the electrode rehydrator 414 or output of the electrostimulation signal from the electrode interface 424. In an example, the electrode rehydrator 414 can be in wireless communication with the electrostimulation electronics unit 404 such as to communicate instructions between, e.g., the electrode rehydrator and the processor circuitry 420.

Communications between the electrode rehydrator 414 and the electrostimulation electronics unit 404 are described in greater detail below.

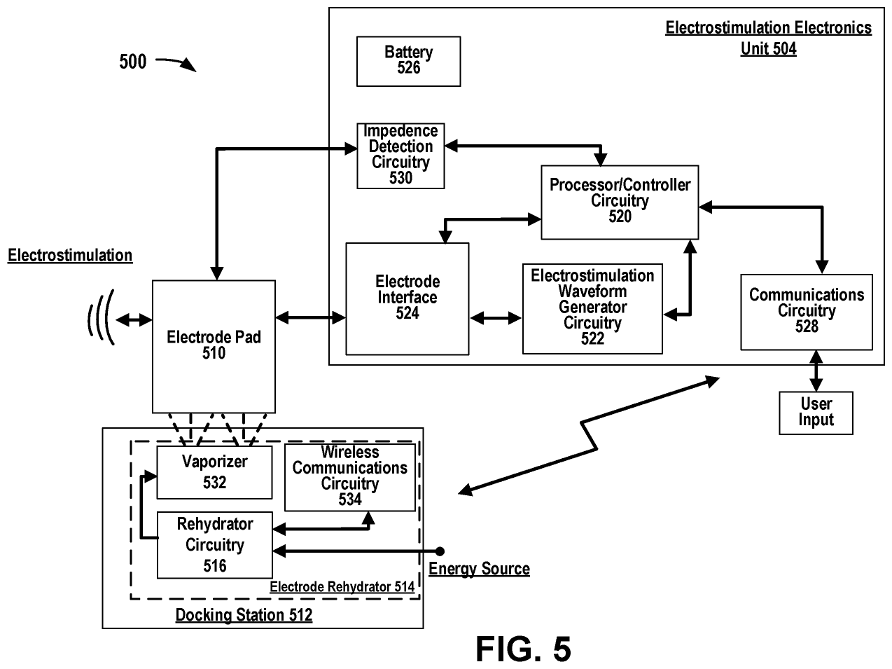

FIG. 5 is a schematic representation of an example of a transcutaneous neurostimulation therapy system. An example electrostimulation therapy system 500 can include an electrostimulation electronics unit 504, one or more electrode pads 510, a docking station 512 and an electrode rehydrator 514. The electrostimulation therapy system 500 can be similar in many respects to electrostimulation therapy systems 100 and 400. The components, structures, configuration, functions, etc. of system 500 can therefore be the same as or substantially similar to that described in detail above with reference to systems 100 and 400. Similar to that previously described with respect to system 400, system 500 can include the electrostimulation electronics unit 504 capable of communication with either of the docking station 512 or the electrode rehydrator 514. The electrostimulation electronics unit 504 can include or use a processor or controller circuitry 520, an electrostimulation waveform generator circuitry 522, an electrode interface 524, and a battery 526. The electrostimulation electronics unit 504 can also include communications circuitry 528 and impedance detection circuitry 530. Also, the electrode rehydrator 514 can include or use or be coupled to a vaporizer 532, rehydrator circuitry 534, and wireless communications circuitry 534. The system 500 can also include or use a system controller communicatively coupled to one or more system components. In an example, any of the vaporizer 532, rehydrator circuitry 534, and wireless communications circuitry 534 can be at least partially disposed or contained within the docking station 512. The wireless communications circuitry 534 of the electrode rehydrator 514 can communicate wirelessly with communications circuitry 528 of the electrostimulation electronics unit 504 such as to exchange instructions between the two. In an example, the communications circuitry 528 can also be wirelessly or electrically coupled to a user interface (UI) such as an onboard UI or software for providing user input from a remote device. Wireless communication can be a BLUETOOTH connection, a Wi-Fi connection, a cellular connection, a near-field communication (NFC) connection, a radiofrequency (RF) connection, or combinations thereof. In an example, the wireless communications circuitry 534 or the communications circuitry 528 can include or use a BLUETOOTH chip capable of broadcasting e.g., in the 2.4 GHz industrial, scientific, and medical (ISM) radio band. In another example, the electrode rehydrator 514 can communicate with the communications circuitry 528 or the processor circuitry 520 through a wired connection, such as a USB connection. In another example, the electrode rehydrator 514 can communicate with the communications circuitry 528 or the processor circuitry 520 through the one or more electrode pads, such as through neurostimulation output terminals. Here, the electrode rehydrator 514 can be capable of generating a communications signal to be received by e.g., the electrode interface 524 of the electrostimulation electronics unit 504. The communications signal can be modulated by the electrode rehydrator 514, such as through amplitude modulation (AM), frequency modulation (FM), or pulse-width modulation (PWM).

The communications between the electrostimulation electronics unit 504 and the electrode rehydrator 514 can include instructions which can be read by either of the processor circuitry 420 or the rehydrator circuitry 516 to control operations of the electrostimulation output of the electrostimulation electronics unit 504 or a rehydration output of the electrode rehydrator, respectively. In an example, either of the communications circuitry 528 or the wireless communications circuitry 534 can be capable of determining the proximity of the electrode rehydrator 514 and the electrostimulation electronics unit 504. Where the two have at most a predetermined proximity to each other, the communications circuitry 528 or the wireless communications circuitry 534 can deliver instructions for either of the electrostimulation electronics unit 504 or the electrode rehydrator 514 to enter a rehydrating mode. In the rehydrating mode, the electrode rehydrator can be selectively operated by, e.g., the rehydrator circuitry 516 such as to distribute moisture to the one or more electrode pads. Also, in the rehydrating mode, either of the electrostimulation waveform generator circuitry 522 or the electrode interface 524 can be selectively operated by the processor circuitry 520 such as to cease electrostimulation output distributed by the one or more electrode pads 510 during rehydration.

In another example, the communications between the electrostimulation electronics unit 504 and the electrode rehydrator 514 can include instructions which can be read by either of the processor circuitry 420 or the rehydrator circuitry 516 such as to impede one or more system functions until a compliant electrode pad is supplied or replaced in connection with the electrostimulation electronics unit 504. For example, the processor 520 can impede electrostimulation output until receiving communications or detecting that a compliant electrode pad has been supplied or replaced. Also, the processor 520 can impede battery charging until receiving communications or detecting that a compliant electrode pad has been supplied or replaced. Likewise, the rehydrator circuitry can impede electrode rehydration until receiving communications or detecting that a compliant electrode pad has been supplied or replaced.

Compliance of the one or more electrode pads 110 can be detected by a circuit included within any of the electrostimulation electronics unit 504, the electrode rehydrator 514, or the docking station 512. In an example, the electrode pad 510 can include or use a usability indicator including an initiator such as an NFC chip attached to or embedded within the pad 510 for communication with, e.g., the communications circuitry 528 or the wireless communications circuitry 534. The usability indicator can transmit, e.g., a radiofrequency signal to be read by an indicator reader included in circuitry 528 or 534. Circuitries 528 or 534 can include or use memory including instructions to impede one or more system functions upon detection (or instructions of) an electrode pad without a compliant usability indicator or an electrode pad that has exceeded a predetermined amount of uses in electrostimulation. In another example, the electrostimulation electronics unit 504 can include or use impedance detection circuitry 530 which can be capable of measuring an impedance or resistance across the one or more electrode pads 510. The impedance detection circuitry 530 can be communicatively coupled to the processor circuitry 520. In an example, circuitries 528 or 534 can include or use memory including instructions to impede one or more system functions upon detection (or instructions of) an electrode pad 510 having an impedance deviating from the expected operating conditions and which is indicative of deteriorated electrode pad electrical performance. In an example, the impedance detection circuitry 530 provides a classifier signal indicative of electrode pad impedance exceeding 10% deterioration from new-electrode-pad impedance. Impedance of the electrode pad 510 measured by the impedance detection circuitry 530 can also be detected by or communicated to the rehydrator circuitry 516 such as to influence selective operation of the rehydration provided by the electrode rehydrator 514. In an example, the rehydrator circuitry 516 can include or use memory including instructions to provide electrode rehydration upon detection (or instructions of) an electrode pad 510 having an impedance deviating from the expected operating conditions and which is indicative of deteriorated electrode pad electrical performance or an impedance exceeding 10% deterioration from new-electrode-pad impedance. In an example, the impedance of the electrode pad 510 can be recurrently, periodically, or continually monitored by the impedance detection circuitry 530 during electrode rehydration such as to selectively operate the electrode rehydrator 514. Rehydration of the electrode pad 510 can progressively lower a measured impedance, and impedance monitoring can help the system 500 reach or maintain a desired condition of the electrode pads 510 before electrostimulation therapy. The electrode rehydrator 514, the docking station 512, or the electrostimulation electronics unit 504 can also include other components capable of measuring or estimating moisture content or saturation measuring of the electrode pad 510 such as to help selectively operate the electrode rehydrator to help the system 500 reach or maintain a desired condition of the electrode pads 510 before electrostimulation therapy. Alternative or additional components for measuring or estimating moisture content or saturation can include moisture meters or hygrometers.

The electrostimulation therapy system can include or use a system controller, and the system controller can be located within or be connected to the processor/controller circuitry or the rehydrator circuitry, or the system controller can be remotely located. System controller can include hardware, software, and combinations thereof to implement the functions attributed to the controller herein. System controller can be an analog, digital, or combination analog and digital controller including a number of components. As examples, controller can include ICB(s), PCB(s), processor(s), data storage devices, switches, relays, etcetera. Examples of processors can include any one or more of a microprocessor, a controller, a digital signal processor (DSP), an application specific integrated circuit (ASIC), a field-programmable gate array (FPGA), or equivalent discrete or integrated logic circuitry. Storage devices, in some examples, are described as a computer-readable storage medium. In some examples, storage devices include a temporary memory, meaning that a primary purpose of one or more storage devices is not long-term storage. Storage devices are, in some examples, described as a volatile memory, meaning that storage devices do not maintain stored contents when the computer is turned off. Examples of volatile memories include random access memories (RAM), dynamic random-access memories (DRAM), static random-access memories (SRAM), and other forms of volatile memories known in the art. The data storage devices can be used to store program instructions for execution by processor(s) of controller. The storage devices, for example, are used by software, applications, algorithms, as examples, running on and/or executed by controller. The storage devices can include short-term and/or long-term memory and can be volatile and/or non-volatile. Examples of non-volatile storage elements include magnetic hard discs, optical discs, floppy discs, flash memories, or forms of electrically programmable memories (EPROM) or electrically erasable and programmable (EEPROM) memories.

System controller can be configured to communicate with the electrostimulation therapy system 500 and components thereof via various wired or wireless communications technologies and components using various public and/or proprietary standards and/or protocols. For example, a power and/or communications network of some kind may be employed to facilitate communication and control between controller and electrostimulation therapy system 500. In one example, system controller can communicate with electrostimulation therapy system 500 via a private or public local area network (LAN), which can include wired and/or wireless elements functioning in accordance with one or more standards and/or via one or more transport mediums.

In one example, controller and electrostimulation therapy system 500 can be configured to use wireless communications according to one of the 802.11 or Bluetooth specification sets, or another standard or proprietary wireless communication protocol. Data transmitted to and from components of electrostimulation therapy system 500, including controller, can be formatted in accordance with a variety of different communications protocols. For example, all or a portion of the communications can be via a packet-based, Internet Protocol (IP) network that communicates data in Transmission Control Protocol/Internet Protocol (TCP/IP) packets, over, for example, Category 5, Ethernet cables.

System controller can selectively operate several components in example electrostimulation systems included herein. System controller can include one or more programs, circuits, algorithms, or other mechanisms for controlling the operation of electrostimulation therapy system 500. For example, the system controller can be configured to selectively operate an electrode rehydrator, electrostimulation waveform generator circuitry, signal modulators, or other components of electrostimulation therapy systems described herein.

In operation and use, an electrostimulation electronics unit can be provided or obtained. One or more skin electrode pads can be attached to or paired with at least one electrostimulation output terminal, and the electrostimulation electronics unit can be selectively operated in a therapy mode such as to provide electrostimulation therapy to a subject. An electrode rehydrator can also be provided or obtained, and the electrode rehydrator can idle or reduce rehydrating while the electrostimulation electronics unit is selectively operated in the therapy mode. In an example, the electrostimulation electronics unit and the electrode rehydrator can be selectively operated to enter a rehydrating mode wherein the electrode rehydrator supplies moisture to the at least one electrode pad. The electrode rehydrator can be selectively operated to supply the moisture based on any of the moisture content of the pad, the impedance of the pad, or the compliance of an indicator attached to or embedded within the pad. The electrostimulation electronics unit can be selectively operated such that an electrostimulation output is impeded while the electrode rehydrator is supplying the moisture in the rehydrating mode.

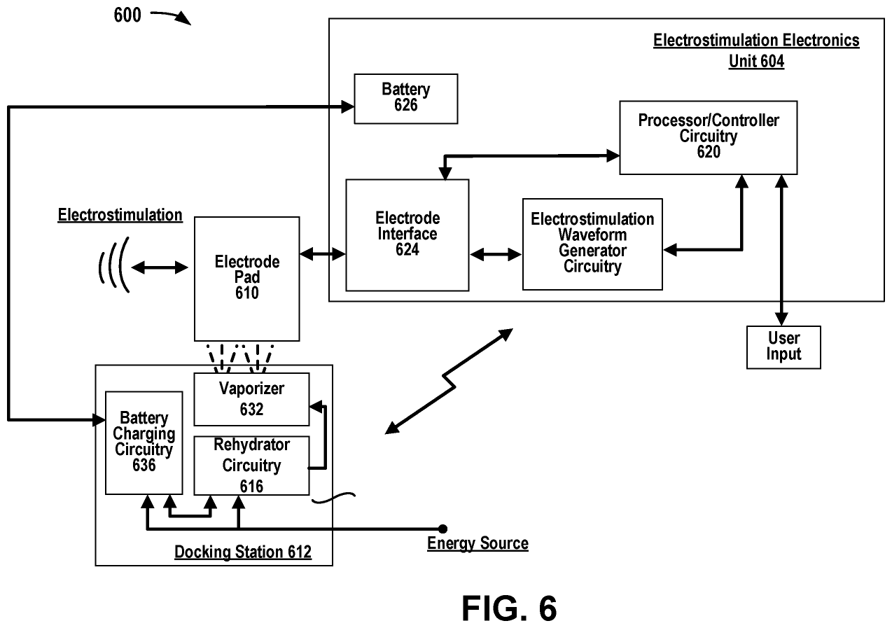

FIG. 6 is a schematic representation of an example of a transcutaneous neurostimulation therapy system. An example electrostimulation therapy system 600 can include an electrostimulation electronics unit 604, electrode terminals 608, electrode pads 610, a docking station 612, rehydrator circuitry 616, and battery charging circuitry 636. The electrostimulation therapy system 600 can be similar in many respects to electrostimulation therapy systems 100, 400, and 500.

The components, structures, configuration, functions, etc. of system 600 can therefore be the same as or substantially similar to that described in detail above with reference to systems 100, 400, and 500. Battery charging can be supplied along with rehydration from the vaporizer 632 at the docking station 612, such as concurrently or at least during the same docking session. Charge-receiving electrical contacts of the electrostimulation electronics unit 604 can be coupled to the charge-supplying electrical contacts electrically connected to the battery charging circuitry 636 for supplying battery charging. Also, the docking station 612 can include or use a wireless power transmission (WPT) transmitter and the electrostimulation electronics unit 604 can include or use a wireless power transmission (WPT) receiver. For instance, the docking station 612 and the electrostimulation electronics unit 604 can each include induction coils such as to enable wireless charging through electromagnetic inductive coupling. The battery charging circuitry 636 can also interface with the electrostimulation electronics unit 604 at the docking station 612 by wired mechanical connections such as cables and receptacles.

The battery charging circuitry 636 can be at least partially disposed within a docking station 412. The battery charging circuitry 636 can be connected to an energy source. For instance, the electrostimulation electronics unit 604 can include or use circuitry for controlling distribution of the charging signal to the battery 626. The battery charging circuitry 636 can be capable of communication with the electrostimulation electronics unit 604 such as to help moderate battery charging from the battery charging circuitry 636 or output of the electrostimulation signal from the electrode interface 624. In an example, the battery charging circuitry 636 can be in wireless communication with the electrostimulation electronics unit 604 such as to communicate instructions between, e.g., the battery charging circuitry 636 and the processor circuitry 620.

The charging signal supplied by the battery charging circuitry 636 can be a symmetrical AC waveform. The voltage regulator can be included in the electrostimulation electronics unit 604 such as to regulate the voltage of the charging signal to a suitable voltage for charging the battery 626. The voltage regulator can regulate the voltage of the charging signal to be relatively close to the voltage of the battery. For instance, the voltage regulator can regulate the voltage of the charging signal to be within a range between about 3V to about 5V. The voltage regulator can regulate the voltage of the charging signal to be about 4.2V Also, the voltage regulator can regulate the voltage of the charging signal to near a predetermined intermediate voltage between the voltage of the charging signal supplied by the battery charging circuitry 636 and a suitable voltage for charging the battery 626. For instance, the voltage regulator can regulate the voltage of the charging signal to be within a range of about 4.4V to about 10V. The voltage regulator 646 can regulate the voltage of the charging signal to be about 4.5V. The voltage regulator can also stabilize the voltage of the charging signal such as to minimize voltage fluctuation prior to delivery to the battery 626 for charging. The voltage regulator can pass the voltage-regulated, AC charging signal to an AC-to-DC converter in the electrostimulation electronics unit 604, and the AC-to-DC converter can convert the AC waveform to a DC waveform for charging the battery 626. The AC-to-DC converter can include or use a rectifier, power supply unit (PSU), switched-mode power supply, or combinations thereof. The battery 626 can be any type of rechargeable battery such as Lithium-ion battery (Li-ion), nickel-cadmium (NiCd), nickel-metal hydride (NiMH), or Lithium-ion polymer (LiPo). The battery 626 can include or use one or more DC components for applying direct current.

In an example, battery charging circuitry 636 can communicate with the processor circuitry 620 through the charge-supplying electrical contacts and the charge-receiving electrical contacts. Here, the battery charging circuitry can be capable of transmitting a communications signal through the charging conduit such as to be received by the electrostimulation electronics unit 704. Also, the battery charging circuitry 636 can encode data onto the charging signal to be received by the electrostimulation electronics unit 604. For instance, a signal encoder can be included in or used by the battery charging circuitry 636 such as to encode data onto the waveform of the charging signal. The waveform of the charging signal can be modulated by the signal encoder, such as through amplitude modulation (AM), frequency modulation (FM), or pulse-width modulation (PWM). The electrostimulation electronics unit 604 can include or use the signal decoder such as for decoding data encoded onto the charging signal waveform. The signal decoder can be communicatively coupled with the processor circuitry 620 such as for transmitting communications thereto. The electrostimulation electronics unit 604 can also encode data onto the charging signal to be received by the battery charging circuitry 636. For example, the electrostimulation electronics unit 604 can include or use or be coupled to a load encoder, and the load encoder can modulate data into the charging signal such as by changing or interrupting the load current. For instance, the load encoder can briefly interrupt charging of the battery or briefly draw extra load such as to modulate data into the charging signal. Also, the battery charging circuitry 636 can include or use or be coupled to the load decoder such as for decoding data encoded onto the charging signal waveform. For example, the load decoder can include or use a current measurement circuit for detecting modulation of the current such as by the load encoder.

The communications between the electrostimulation electronics unit 604 and the battery charging circuitry 636 can include instructions which can be read by either of the processor circuitry 620 or the battery charging circuitry 636 such as to impede one or more system functions until a compliant electrode pad 610 is supplied or replaced in connection with the electrostimulation electronics unit 604. For example, the processor circuitry 620 can impede electrostimulation output until receiving communications or detecting that a compliant electrode pad 610 has been supplied or replaced. Also, the battery charging circuitry 636 can impede battery charging until receiving communications or detecting that a compliant electrode pad 610 has been supplied or replaced. Likewise, the battery charging circuitry 636 can impede battery charging until receiving communications or detecting that a compliant electrode pad 610 has been supplied or replaced. Compliance of the one or more electrode pads 610 can be detected by a circuit located in any of the electrostimulation electronics unit 604, the battery charging circuitry 636, or the docking station 612.

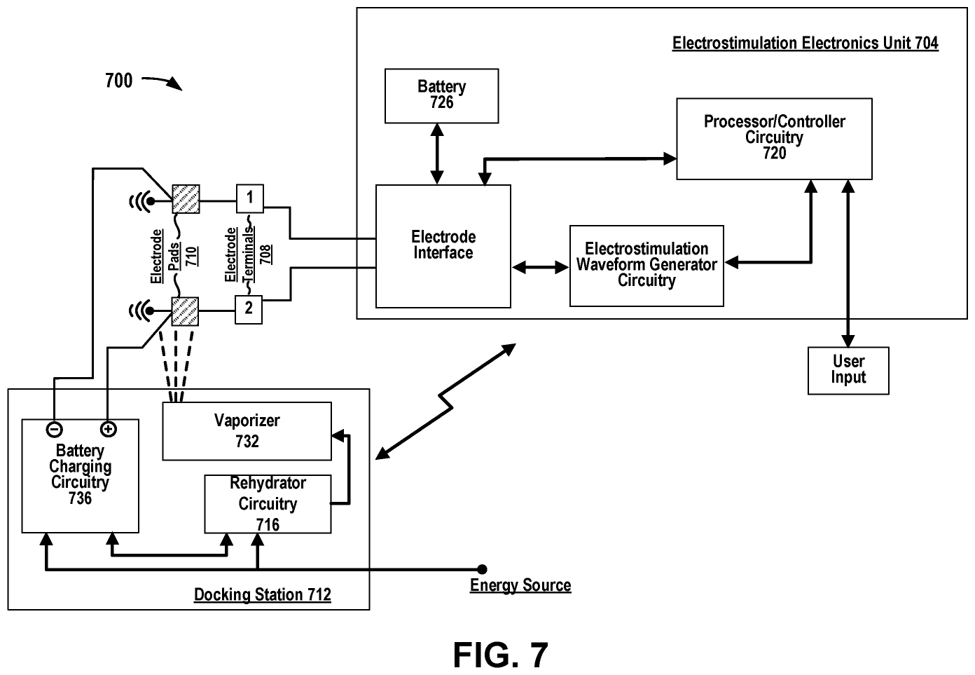

FIG. 7 is a schematic representation of an example of a transcutaneous neurostimulation therapy system. An example electrostimulation therapy system 700 can include an electrostimulation electronics unit 704, electrode terminals 708, electrode pads 710, a docking station 712, rehydrator circuitry 716, and battery charging circuitry 736. The electrostimulation therapy system 700 can be similar in many respects to electrostimulation therapy systems 100, 400, 500, and 600. The components, structures, configuration, functions, etc. of system 700 can therefore be the same as or substantially similar to that described in detail above with reference to systems 100, 400, and 500, and 600. Battery charging can be supplied along with rehydration from the vaporizer 732 at the docking station 712, such as concurrently or at least during the same docking session. Electrode terminals 708 of the electrostimulation electronics unit 704 can be coupled through the electrode pads 710 and to the charge-supplying electrical contacts. The charge-supplying electrical contacts can carry a charging signal generated by charging circuitry 736 for supplying battery charging.

In an example where the battery charging circuitry 736 can charge the battery of the electrostimulation electronics unit 704 through the electrode pads 710 while the electrode pads 710 are concurrently rehydrated by the electrode rehydrator 714, the docking station 712 can provide an electrically isolated barrier between electrode pads 710. For example, the docking station can provide an electrically isolated barrier between charge-supplying electrical contacts, and each electrode pad 710 can be sized and shaped such as to be placed in an electrically isolated receptacle created by the barrier. The electrode terminals 708 can each include capacitive coupling (e.g., coupled using series DC-blocking capacitors, e.g., in a charge-balanced arrangement) between the battery of the electrostimulation electronics unit, and the battery charging circuitry can be configured to be capacitively coupled via the two or more output terminals to the electrostimulation electronics unit for battery charging. In an example, battery charging circuitry 736 can communicate with the processor circuitry 720 through the one or more electrode pads, such as through neurostimulation output terminals.

The following, non-limiting examples, detail certain aspects of the present subject matter to solve the challenges and provide the benefits discussed herein, among others.

Example 1 is a neurostimulation system, comprising: a skin electrode pad configured to be attached to an external electrostimulation electronics unit for treating a subject using transcutaneous neurostimulation therapy, the electrode pad comprising: a subject skin contact surface; a rehydratable hydrophilic foam layer configured to be disposed between a neurostimulation output terminal of the electrostimulation electronics unit and the subject skin contact surface; and an electrolyte carried by the hydrophilic foam layer; and wherein the rehydratable hydrophilic foam layer is sized and shaped to be received within a receptacle of an electrode rehydrator to recurrently receive moisture from the electrode rehydrator to be rehydrated.

In Example 2, the subject matter of Example 1, wherein the rehydratable hydrophilic foam layer comprises polyvinyl alcohol (PVA).

In Example 3, the subject matter of any of Examples 1-2, wherein the rehydratable hydrophilic foam layer is configured to recurrently receive a vapor from a vaporizer configured to vaporize a liquid and to supply the vapor to the rehydratable hydrophilic foam layer.

In Example 4, the subject matter of any of Examples 1-3, wherein the rehydratable hydrophilic foam layer has a thickness-to-pore-diameter ratio of at least ten to one.

In Example 5, the subject matter of any of Examples 1-4, wherein the rehydratable hydrophilic foam layer includes an absorbent material having a water contact angle ranging between forty five degrees and fifty five degrees.

In Example 6, the subject matter of any of Examples 1-5, wherein the rehydratable hydrophilic foam layer includes a polymer chain with polar end groups.

In Example 7, the subject matter of any of Examples 1-6, further comprising a substrate layer disposed between the neurostimulation output terminal of the electrostimulation electronics unit and the rehydratable hydrophilic foam layer.

In Example 8, the subject matter of Example 7, wherein the substrate layer includes one or more electrically conductive traces.

In Example 9, the subject matter of Example 8, wherein the electrically conductive traces include conductive ink printed on the substrate.

In Example 10, the subject matter of any of Examples 1-9, further comprising the electrode rehydrator, and wherein: the electrode rehydrator is configured to supply moisture to recurrently rehydrate the neurostimulation skin electrode pad.

In Example 11, the subject matter of Example 10, further comprising the electrostimulation electronics unit, and wherein: the electrostimulation electronics unit is configured to be attached the neurostimulation skin electrode pad, and the electrostimulation electronics unit comprises: at least one neurostimulation output terminal configured for delivering neurostimulation to a subject via the at least one neurostimulation skin electrode pad.

In Example 12, the subject matter of any of Examples 10-11, further comprising a system controller configured to control water transfer from the electrode rehydrator and to the electrode pad.

In Example 13, the subject matter of Example 12, wherein the system controller is configured to impede water transfer upon detection of a moisture content of the electrode pad being greater than a predetermined value.

In Example 14, the subject matter of Example 13, wherein the system controller is configured to impede water transfer upon detection of an electrical impedance of the electrode pad being less than a predetermined value.

In Example 15, the subject matter of Example 14, wherein the system controller is configured to impede water transfer upon detection of a moisture content from a rehydrator circuitry of the electrode rehydrator being greater than a predetermined value.

In Example 16, the subject matter of any of Examples 10-15, wherein the electrode rehydrator includes or is connected to a vaporizer configured to vaporize a liquid and to supply vapor to the electrode pad.

In Example 17, the subject matter of Example 16, wherein the vaporizer includes an ultrasonic mister.

In Example 18, the subject matter of Example 17, wherein the ultrasonic mister includes a ceramic diaphragm configured to vibrate the liquid.

In Example 19, the subject matter of any of Examples 10-18, wherein the electrode rehydrator includes a reservoir configured to supply liquid directly to the electrode pad.

In Example 20, the subject matter of any of Examples 10-19, wherein the electrode rehydrator includes or is connected to a battery charging circuitry configured for charging a battery of the electrostimulation electronics unit.

In Example 21, the subject matter of Example 20, wherein the battery charging circuitry is configured to charge the battery through at least one neurostimulation output terminal that is configured for delivering neurostimulation to a subject.

Example 22 is a neurostimulation system, comprising: at least one of an electrostimulation electronics unit or an electrode rehydrator, comprising: at least one neurostimulation output terminal configured for delivering neurostimulation to a subject via a neurostimulation an electrode pad including a hydrophilic foam layer; and moisture detection circuitry configured to detect a moisture content of the hydrophilic foam layer of the electrode pad.

In Example 23, the subject matter of Example 22, wherein the moisture detection circuitry is configured for measuring an electrical impedance across the hydrophilic foam layer.

Example 24 is an electrode docking station configured to supply moisture to recurrently rehydrate an electrostimulation skin electrode pad of an electrostimulation electronics unit, the electrode docking station comprising: a vaporizer configured to vaporize liquid water and to supply water vapor to the electrode pad; rehydrator circuitry configured connected to the vaporizer and configured for controlling an amount of water vapor supplied to the electrode pad; and battery charging circuitry configured for charging a battery of the electrostimulation electronics unit.

In Example 25, the subject matter of Example 24, wherein the vaporizer is an ultrasonic mister.

In Example 26, the subject matter of any of Examples 24-25, wherein the ultrasonic mister includes a ceramic diaphragm configured to vibrate liquid water.

In Example 27, the subject matter of any of Examples 24-26, further comprising moisture detection circuitry configured to detect a moisture content of the hydrophilic foam layer of the electrode pad.

Example 28 is a method of preparing a transcutaneous neurostimulation device, the method comprising: providing or obtaining an neurostimulation skin electrode pad, configured to be electrically coupled to at least one neurostimulation output terminal for delivering neurostimulation to a subject; and providing or obtaining impedance detection circuitry configured to detect an impedance of the neurostimulation skin electrode pad; providing or obtaining an electrode rehydrator, the rehydrator configured to selectively operate to supply moisture to recurrently rehydrate the neurostimulation skin electrode pad; and selectively operating the electrode rehydrator based on the impedance of the neurostimulation skin electrode pad detected by the moisture detection circuitry.

Example 29 is a neurostimulation system, comprising: a neurostimulation skin electrode pad configured to be attached to an electrostimulation electronics unit for treating a subject using transcutaneous neurostimulation therapy, the electrode pad comprising: a subject skin contact surface; a rehydratable hydrophilic foam layer configured to be disposed between a neurostimulation output terminal of the electrostimulation electronics unit and the subject skin contact surface; and an electrolyte carried by the hydrophilic foam layer; and a neurostimulation skin electrode pad usability indicator configured to be communicated with a usability indicator reader; and wherein the rehydratable hydrophilic foam layer is configured to recurrently receive moisture from an electrode rehydrator to be rehydrated.

In Example 30, the subject matter of Example 29, wherein the usability indicator includes a near-field wireless communications circuitry.

In Example 31, the subject matter of any of Examples 29-30, wherein the usability indicator includes an initiator configured to generate and radiofrequency (RF) field to be communicated with a target of the usability indicator reader.

Example 32 is at least one machine-readable medium including instructions that, when executed by processing circuitry, cause the processing circuitry to perform operations to implement of any of Examples 1-31.

Example 33 is an apparatus comprising means to implement of any of Examples 1-31.

Example 34 is a system to implement of any of Examples 1-31.

Example 35 is a method to implement of any of Examples 1-31.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A neurostimulation system, comprising:
a skin electrode pad configured to be attached to an external electrostimulation electronics unit for treating a subject using transcutaneous neurostimulation therapy, the electrode pad comprising:
a subject skin contact surface;
a rehydratable hydrophilic foam layer configured to be disposed between a neurostimulation output terminal of the electrostimulation electronics unit and the subject skin contact surface; and
an electrolyte carried by the hydrophilic foam layer; and
wherein the rehydratable hydrophilic foam layer is sized and shaped to be received within a receptacle of an electrode rehydrator to recurrently receive moisture from the electrode rehydrator to be rehydrated.

2. The system of claim 1, wherein the rehydratable hydrophilic foam layer comprises polyvinyl alcohol (PVA).

3. The system of claim 1, wherein the rehydratable hydrophilic foam layer is configured to recurrently receive a vapor from a vaporizer configured to vaporize a liquid and to supply the vapor to the rehydratable hydrophilic foam layer.

4. The system of claim 1, wherein the rehydratable hydrophilic foam layer has a thickness-to-pore-diameter ratio of at least ten to one.

5. The system of claim 1, wherein the rehydratable hydrophilic foam layer includes an absorbent material having a water contact angle ranging between forty five degrees and fifty five degrees.

6. The system of claim 1, wherein the rehydratable hydrophilic foam layer includes a polymer chain with polar end groups.

7. The system of claim 1, further comprising a substrate layer disposed between the neurostimulation output terminal of the electrostimulation electronics unit and the rehydratable hydrophilic foam layer.

8. The system of claim 7, wherein the substrate layer includes one or more electrically conductive traces.

9. (canceled)

10. The system of claim 1, further comprising the electrode rehydrator, and wherein:
the electrode rehydrator is configured to supply moisture to recurrently rehydrate the neurostimulation skin electrode pad.

11. The system of claim 10, further comprising the electrostimulation electronics unit, and wherein:
the electrostimulation electronics unit is configured to be attached the neurostimulation skin electrode pad, and the electrostimulation electronics unit comprises: at least one neurostimulation output terminal configured for delivering neurostimulation to a subject via the at least one neurostimulation skin electrode pad.

12. The system of claim 10, further comprising a system controller configured to control water transfer from the electrode rehydrator and to the electrode pad.

13. The system of claim 12, wherein the system controller is configured to impede water transfer upon detection of a moisture content of the electrode pad being greater than a predetermined value.

14. The system of claim 13, wherein the system controller is configured to impede water transfer upon detection of an electrical impedance of the electrode pad being less than a predetermined value.

15. The system of claim 14, wherein the system controller is configured to impede water transfer upon detection of a moisture content from a rehydrator circuitry of the electrode rehydrator being greater than a predetermined value.

16. The system of claim 10, wherein the electrode rehydrator includes or is connected to a vaporizer configured to vaporize a liquid and to supply vapor to the electrode pad.

17. (canceled)

18. The system of claim 17, wherein the vaporizer includes an ultrasonic mister including a ceramic diaphragm configured to vibrate the liquid.

19. The system of claim 10, wherein the electrode rehydrator includes a reservoir configured to supply liquid directly to the electrode pad.

20. (canceled)

21. The system of claim 20, wherein the battery charging circuitry is configured to charge the battery through at least one neurostimulation output terminal that is configured for delivering neurostimulation to a subject.

22. A neurostimulation system, comprising:
at least one of an electrostimulation electronics unit or an electrode rehydrator, comprising:
at least one neurostimulation output terminal configured for delivering neurostimulation to a subject via a neurostimulation an electrode pad including a hydrophilic foam layer; and
moisture detection circuitry configured to detect a moisture content of the hydrophilic foam layer of the electrode pad.

23. The neurostimulation system of claim 22, wherein the moisture detection circuitry is configured for measuring an electrical impedance across the hydrophilic foam layer.

24. An electrode docking station configured to supply moisture to recurrently rehydrate an electrostimulation skin electrode pad of an electrostimulation electronics unit, the electrode docking station comprising:
a vaporizer configured to vaporize liquid water and to supply water vapor to the electrode pad;
rehydrator circuitry configured connected to the vaporizer and configured for controlling an amount of water vapor supplied to the electrode pad; and
battery charging circuitry configured for charging a battery of the electrostimulation electronics unit.

25. (canceled)

26. The docking station of claim 24, wherein the ultrasonic mister includes a ceramic diaphragm configured to vibrate liquid water.

27-31. (canceled)
